(19) United States
(12) Patent Application Publication
(10) Pub. No.: US 2026/0256467 A1
(43) Pub. Date: Sep. 3, 2026

(54) SEMI-TUBULAR OPEN RETRACTOR SYSTEMS FOR MULTI-PORTAL ENDOSCOPIC SPINAL SURGERY

(71) Applicant: Samuel Cho, Englewood Cliffs, NJ (US)

(72) Inventor: Samuel Cho, Englewood Cliffs, NJ (US)

(73) Assignee: ChoSpine, LLC, Englewood Cliffs, NJ (US)

(21) Appl. No.: 19/657,531

(22) Filed: Apr. 24, 2026

Related U.S. Application Data
(63) Continuation-in-part of application No. 19/418,535, filed on Dec. 12, 2025.
(60) Provisional application No. 63/733,009, filed on Dec. 12, 2024.

(51) Int. Cl. A61B 17/02; A61B 17/00; A61B 17/56; A61B 34/10; A61B 34/30; A61B 90/00; A61B 90/30; A61F 2/44; A61F 2/46

(52) U.S. Cl. CPC A61B 17/025; A61B 17/56; A61B 34/10; A61B 34/30; A61B 90/30; A61B 90/39; A61F 2/44; A61F 2/4611; A61B 2017/00039; A61B 2017/00544; A61B 2017/00561; A61B 2017/0092; A61B 2017/0256; A61B 2017/564; A61B 2090/309; A61B 2090/3966; A61B 2217/005; A61F 2002/4633

(57) ABSTRACT

A non-expandable surgical retractor having a central working channel of internal diameter D_i and an open sector defined by a solid wall arc between about 180 degrees and about 300 degrees, extending along at least 50% of the body length, the open sector having a transverse chord width W between two longitudinal edges geometrically determined by the arc angle. The open sector forms a region extending radially beyond the outer circumference without intervening structure, permitting a portion of a spinal implant with maximum transverse dimension D_IMPL satisfying D_i<D_IMPL and D_IMPL less than or equal to D_i+W to occupy the open sector during passage—without mechanical expansion. Integrated features include: modular obturator; compliant sheath; robotic angular alignment relative to targeted anatomical structures using anatomical imaging data; illumination; fluid evacuation; static retraction mechanism; directional neuromonitoring; anti-migration docking surface; and asymmetric radiopaque orientation markers.

### CROSS-REFERENCE TO RELATED APPLICATIONS

This application is a Continuation-in-Part of U.S. patent application Ser. No. 19/418,535, filed on Dec. 12, 2025, which claims the benefit of U.S. Provisional Application No. 63/733,009, filed on Dec. 12, 2024. The entire disclosures of the aforementioned applications are incorporated herein by reference in their entirety.

### FIELD OF THE INVENTION

The present disclosure relates to minimally invasive spinal surgical procedures, and more particularly to non-expandable surgical retractor systems for use in bi-portal or multi-portal endoscopic spine surgery. Specifically, the disclosure relates to retractors having a fixed-geometry open sector—defined by a chord-width-to-internal-diameter ratio and a specific arc range—that structurally permits surgical instruments and spinal implants having cross-sectional dimensions exceeding the internal working channel diameter to pass through a percutaneous working portal without any mechanical expansion of the retractor walls.

### BACKGROUND OF THE INVENTION

Minimally invasive spinal surgery (MISS) techniques aim to reduce surgical morbidity by minimizing disruption of surrounding soft tissues, paraspinal muscles, and ligaments. Procedures such as spinal decompression, discectomy, and interbody fusion are increasingly performed through small percutaneous incisions of approximately 0.5 cm to 3.5 cm.

In multi-portal endoscopic techniques, a first percutaneous incision (the “viewing portal”) receives an endoscope; a second incision (the “working portal”) receives surgical instruments and implants. The working portal can be aligned along the trajectory established for percutaneous pedicle screw fixation, sharing a pre-existing corridor to minimize additional tissue trauma.

Conventional fully closed tubular retractors impose a fixed inner diameter that limits the maximum cross-sectional dimensions of passing instruments and implants. Expandable retractors mechanically force their walls outward, transmitting direct radial force to surrounding paraspinal musculature and neurovascular structures and causing iatrogenic injury. When a surgeon needs to deliver an interbody fusion device exceeding the retractor inner diameter, the options are clinically undesirable: remove the retractor and risk unprotected tissue trauma; accept a smaller implant with inferior biomechanical performance; extend the percutaneous incision; or accept the tissue damage of an expandable retractor.

Open or semi-cylindrical instruments are known in other surgical disciplines. However, none of these prior instruments define a chord-width (W)-to-internal-diameter (D_i) geometric relationship sufficient to accommodate partial occupation of the open sector by an implant whose maximum transverse dimension exceeds D_i during longitudinal translation through the instrument. The present disclosure provides a non-expandable retractor system with a precisely defined W/D_i ratio and arc geometry that creates this capability in a device specifically adapted to multi-portal endoscopic spinal surgery.

### SUMMARY OF THE INVENTION

The present disclosure provides a semi-tubular, non-expandable open retractor system. A rigid elongated body defines a central working channel of internal diameter D_i and, by virtue of an incomplete transverse cross-section having a continuous arc between about 180 degrees and about 300 degrees, an open sector between two longitudinal edges. The open sector is bounded by two longitudinal edges separated by a transverse chord width W and extends along at least 50% of the body length. By virtue of the chord-arc geometry of a circular cross-section, W is determined by the arc angle A and the internal diameter D_i according to the relationship W=D_i multiplied by sin((360 degrees minus A) divided by 2), such that W ranges from approximately 0.5 times D_i (at A=300 degrees) to 1.0 times D_i (at A=180 degrees) across the claimed arc range. The open sector forms a region extending radially beyond the outer circumference of the elongated body without intervening structure, allowing a portion of an implant whose maximum transverse dimension D_IMPL satisfies D_i<D_IMPL and D_IMPL is less than or equal to D_i+W to occupy the open sector during passage, without any mechanical actuation of the retractor.

Further aspects of the system encompass: a modular obturator for temporary closed insertion; a compliant sheath—whether removable, yielding, or frangible—positionable over the open sector; a frangible-bridge embodiment; surgical robotic integration configured specifically to orient the open sector at a selected angular position relative to targeted anatomical structures based on anatomical imaging data including preoperative and intraoperative imaging; integrated illumination; fluid evacuation; a tool-securing mechanism for hands-free static retraction; directional neuromonitoring electrodes; a distal bone docking surface; asymmetric radiopaque fluoroscopic orientation markers; and a tapered-diameter embodiment.

### BRIEF DESCRIPTION OF THE DRAWINGS

FIG. 1 is an isometric view of the semi-tubular open retractor showing the open sector extending longitudinally along the elongated body.

FIG. 2A is a transverse cross-sectional view showing: the solid wall arc A (between about 180 degrees and about 300 degrees); the open sector; the internal diameter D_i; the transverse chord width W; and the region extending radially beyond the outer circumference without intervening structure.

FIG. 2B shows three comparative transverse cross-sections at arc=180 degrees, arc=230 degrees (preferred), and arc=300 degrees, with D_i and W labeled to illustrate the W/D_i ratio across the claimed range.

FIG. 2C is a magnified detail callout of one longitudinal edge, taken from FIG. 2A, showing a representative atraumatic edge profile with edge radius r.

FIG. 3A is an in-situ transverse cross-section showing a standard instrument (D_IMPL less than or equal to D_i) fully within the working channel, with surrounding tissue held by the solid arc.

FIG. 3B is an in-situ transverse cross-section showing an oversized implant with D_i<D_IMPL and D_IMPL less than or equal to D_i+W. The portion of the implant occupying the open sector is labeled with dimensional annotations confirming the geometric relationship.

FIGS. 4A and 4B show the modular obturator inserted (continuous 360-degree outer profile) and withdrawn (open sector restored).

FIG. 5 shows the retractor coupled to a robotic arm, with the processor-calculated angular orientation of the open sector labeled relative to identified anatomical structures.

FIGS. 6A and 6B show integrated illumination assembly variations: an optical waveguide within the solid wall (FIG. 6A) and a modular LED at the proximal end (FIG. 6B).

FIG. 7 shows the suction lumen with distal aspiration port and proximal vacuum connector.

FIG. 8 shows the tool-securing longitudinal track with a locked secondary retractor blade maintaining static retraction.

FIG. 9 shows the distal neuromonitoring electrode adjacent to the open sector and the textured anti-migration docking surface.

FIG. 10 shows the radiolucent elongated body with asymmetric radiopaque marker pattern, and the corresponding fluoroscopic projection indicating the rotational orientation of the open sector.

FIGS. 11A-11D show a compliant sheath positioned over the open sector of the elongated body in various configurations: a removable elastomeric sheath in the deployed configuration (FIG. 11A); the sheath in the retracted or removed configuration (FIG. 11B); the sheath yielding laterally or rupturing during implant passage (FIG. 11C); and an alternative frangible film variant that fractures and displaces upon contact with the advancing implant (FIG. 11D).

FIG. 12 shows a tapered embodiment of the elongated body wherein the internal diameter D_i varies along the longitudinal axis from a larger proximal internal diameter to a smaller distal internal diameter, with the arc angle A remaining within about 180 degrees to about 300 degrees along the implant-accommodation region of the open sector.

FIG. 13 shows a transverse cross-sectional view of an alternative embodiment having one or more frangible bridge members spanning the open sector between the first and second longitudinal edges at discrete axial positions.

### DETAILED DESCRIPTION OF THE PREFERRED EMBODIMENTS

The following detailed description enables a person skilled in the art to make and use the invention. The present disclosure is accorded the widest scope consistent with the claims. Specific dimensions and materials described herein are preferred embodiments; the claims are not limited to these specific values unless expressly stated therein.

### I. Definitions

“Open sector” means a region defined between the first and second longitudinal edges of the elongated body, extending radially beyond the outer circumference of the elongated body without intervening structure. The open sector is a fixed geometric feature of the elongated body—it is not created by mechanical actuation and does not change shape under operative loads.

“Transverse chord width (W)” means the straight-line distance between the first and second longitudinal edges measured across the open sector in a transverse plane perpendicular to the longitudinal axis.

“Internal diameter (D_i)” means the diameter of the central working channel, measured as the inner diameter of the solid wall arc portion in a transverse plane.

“Solid wall portion” means the primary structural boundary of the elongated body that defines the internal diameter D_i and the continuous arc profile. The presence of fenestrations, perforations, viewing slots, illumination channels, suction lumens, electrode traces, or secondary instrument ports within this boundary does not preclude the wall from being a “solid wall portion,” provided the overall structural rigidity and the continuous arc profile are substantially maintained. The term refers to structural continuity of the arc, not to an absence of apertures.

“Non-expandable” means the elongated body maintains a substantially fixed cross-sectional geometry under operative loads and does not undergo active mechanical radial expansion during the operative procedure in which the implant is passed through the open sector. This term expressly excludes devices that enlarge their cross-sectional profile through mechanical actuation, hydraulic pressure, pneumatic pressure, thermal expansion, shape-memory actuation, or similar active or passive expansion mechanisms during the operative implant passage. A retractor that possesses independent expansion capabilities but is utilized in a static, unexpanded state—such that implant passage occurs via the fixed geometric clearance of the open sector without mechanical radial expansion during said passage—is non-expandable as used herein with respect to that mode of use.

“Substantially fixed cross-sectional geometry” means that the elongated body does not undergo intentional or designed dimensional change in response to operative loads. The transverse cross-sectional dimensions of the elongated body—including D_i, W, and the arc angle—remain dimensionally stable during operative use, such that any dimensional deviation from nominal unloaded values is attributable solely to incidental elastic deformation under operative loads rather than to any designed expansion mechanism. This definition distinguishes the non-expandable retractor of this disclosure from expandable devices, which are designed to undergo intentional dimensional changes through mechanical actuation, hydraulic pressure, pneumatic pressure, thermal expansion, shape-memory actuation, or similar active or passive expansion mechanisms. A quantitative benchmark for cross-sectional dimensional stability is provided in the preferred embodiments and recited in dependent claims where applicable.

“Intervening structure” means a structural member that permanently and irremovably occludes the open sector such that the structural member prevents the passage of an implant through the open sector during longitudinal translation under all operative conditions. For the purposes of this disclosure, the following do not constitute intervening structure: (a) compliant membranes, yielding sheaths, elastic barriers, frangible films, or destructible coverings—whether removable by the user, integral to the retractor, or designed to rupture or displace upon implant contact—that deform, displace, or rupture upon contact with the implant without impeding longitudinal translation of the implant through the open sector; and (b) rigid or semi-rigid members that are configured to displace, retract, pivot, hinge, rotate, or otherwise move from the open sector to permit implant passage—whether actuated by contact with the implant, by mechanical means, by magnetic means, by gravitational means, or by any other means—provided that such members do not permanently impede longitudinal translation of the implant through the open sector. The defining functional test for intervening structure is whether the member permanently prevents or materially obstructs implant passage through the open sector; any member that permits passage—by yielding, displacing, rupturing, retracting, or moving by any mechanism—is not intervening structure.

“Anatomical imaging data” means imaging data depicting patient anatomy, including without limitation preoperative imaging data (such as preoperative MRI, CT, or radiographic images acquired prior to the surgical procedure) and intraoperative imaging data (such as intraoperative CT, O-arm, cone-beam CT, fluoroscopic, or ultrasound images acquired during the surgical procedure).

“Proximal” means nearest the surgeon during use; “distal” means nearest the surgical target.

“Elongated body” encompasses both a retractor formed from a single, monolithic piece of material and a retractor formed from an assembly of multiple interlocking, modular, or articulable components —such as assembled independent blades, retractor halves, or snap-fit segments—that are rigidly mated or otherwise configured to collectively define the claimed continuous arc and open sector geometric profile when assembled for operative use. Where the claims recite “an elongated body,” the term is satisfied by either a unitary structure or a multi-component assembly that, when assembled, functions as a unitary structural body defining the recited geometric parameters. The “total length of the elongated body” refers to the length of the portion of the elongated body that defines the continuous arc and open sector geometry—it does not include any external handle, robotic mounting interface, proximal extension, or other structure that does not define the arc or open sector and that remains outside the percutaneous surgical access corridor during operative use.

“Maximum transverse dimension (D_IMPL)” of a spinal implant means the maximum cross-sectional dimension of the implant measured in the specific orientation in which the implant is advanced through the elongated body during operative use. This is the dimension that determines whether the implant can be accommodated by the central working channel and open sector geometry. The term does not refer to an absolute maximum diagonal measurement of the implant taken independent of the operative passage orientation—for example, the corner-to-corner diagonal of a rectangular implant measured on a bench is not the “maximum transverse dimension” if the implant is rotated to a different orientation for passage through the retractor.

“Targeted structure” means any anatomical structure identified from anatomical imaging data relative to which the angular orientation of the open sector is determined. Targeted structures include, without limitation, neural structures (such as exiting nerve roots, traversing nerve roots, the thecal sac, and the spinal cord), bony structures (such as the pedicle, lamina, facet joint, and vertebral body endplate), disc structures (such as the annulus fibrosus and nucleus pulposus), and vascular structures. The processor calculates the rotational orientation of the open sector relative to one or more targeted structures to optimize surgical access and safety.

### II. Core Retractor Geometry and the W/D_i Threshold

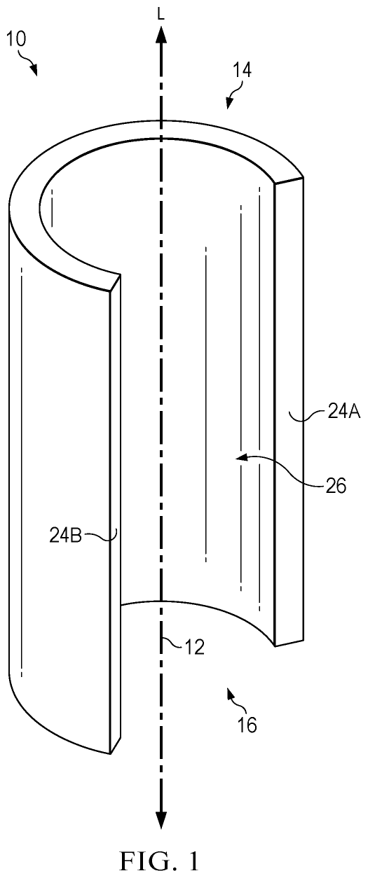

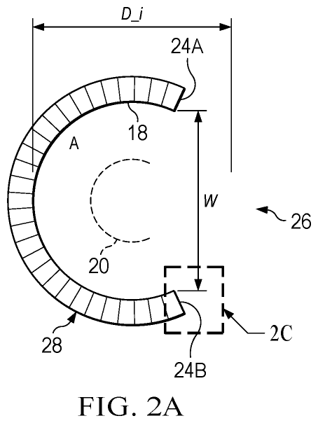

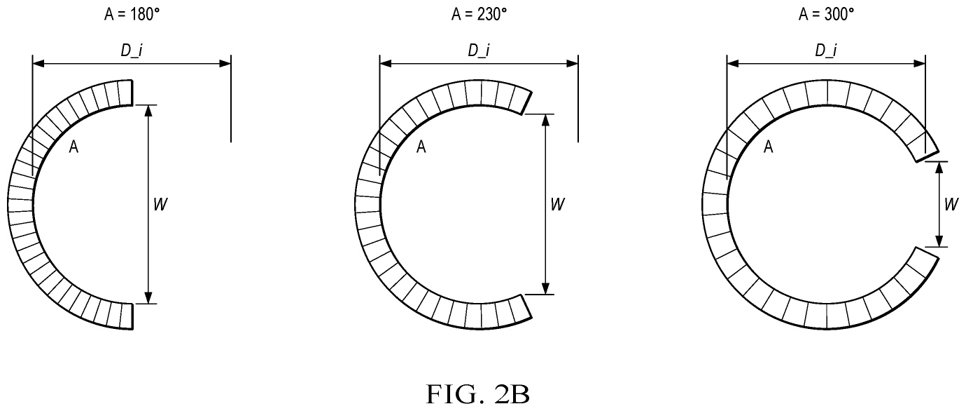

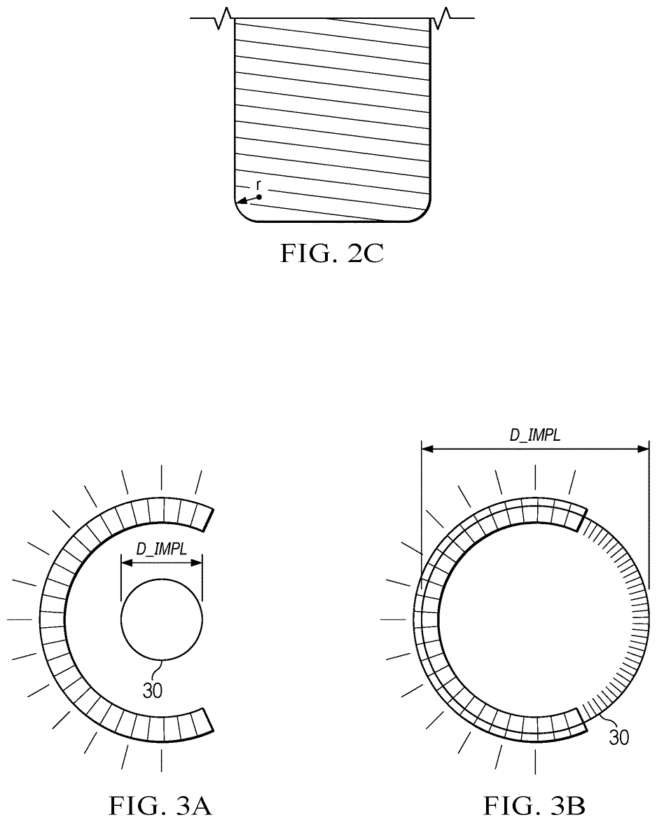

Referring to FIGS. 1 and 2A-2B, the semi-tubular open retractor 10 comprises an elongated, non-expandable body 12 extending along a longitudinal axis L from a proximal end 14 to a distal end 16. In preferred embodiments, the elongated body 12 has a total length of 30 mm to 120 mm and a wall thickness of 0.5 mm to 3.0 mm. The elongated body 12 is formed from a rigid, biocompatible material including, without limitation, surgical-grade stainless steel (e.g., 316L), titanium alloy (e.g., Ti-6Al-4V), cobalt-chromium alloy, PEEK, carbon-fiber-reinforced PEEK, ultra-high-molecular-weight polyethylene (UHMWPE), ceramic composites, or other biocompatible materials suitable for surgical implantation. In fluoroscopy-intensive procedures, PEEK or carbon-fiber-reinforced PEEK may be used to minimize radiographic artifact.

The inner surface 18 defines a central working channel 20 of internal diameter D_i of approximately 5 mm to 35 mm. In a preferred embodiment dimensioned for lumbar or thoracic access, D_i is approximately 10 mm to 20 mm and the outer diameter D_o is approximately 11 mm to 23 mm.

The elongated body 12 terminates circumferentially at a first longitudinal edge 24A and a second longitudinal edge 24B, defining the open sector 26 between them. The solid wall portion 28 spans a continuous arc A of between about 180 degrees and about 300 degrees, and between 200 degrees and 260 degrees in preferred embodiments. The transverse chord width W is geometrically determined by the arc angle A and the internal diameter D_i, as set forth in the following paragraph. In the preferred arc range of 200 degrees to 260 degrees, W accordingly ranges from approximately 0.77 times D_i (at A=260 degrees) to approximately 0.98 times D_i (at A=200 degrees).

Geometric Relationship Between Arc Angle and Chord Width. For a circular cross-section of internal diameter D_i, the transverse chord width W between the two longitudinal edges is geometrically determined by the arc angle A according to the relationship W=D_i multiplied by sin((360 degrees minus A) divided by 2). Worked values across the claimed arc range are as follows: at A=180 degrees, W=D_i multiplied by sin(90 degrees)=1.0 times D_i; at A=240 degrees, W=D_i multiplied by sin(60 degrees), which is approximately 0.866 times D_i; at A=270 degrees, W=D_i multiplied by sin(45 degrees), which is approximately 0.707 times D_i; and at A=300 degrees, W=D_i multiplied by sin(30 degrees)=0.5 times D_i. Across the entire claimed arc range of about 180 degrees to about 300 degrees, W therefore ranges from approximately 0.5 times D_i to 1.0 times D_i. The structural significance of this relationship is that the open sector, by virtue of the arc constraint alone, provides a transverse clearance of at least approximately 0.5 times D_i beyond the inner wall along the chord direction. This clearance permits an implant having a maximum transverse dimension D_IMPL satisfying D_i<D_IMPL and D_IMPL less than or equal to D_i+W to occupy a portion of the open sector during longitudinal translation, without deformation of the implant or the retractor and without tilting the implant out of alignment with the longitudinal axis. The implant-accommodation capability is thus a direct geometric consequence of the arc range itself; no separate chord-width threshold need be imposed beyond the arc constraint. Open or semi-cylindrical instruments known in other surgical fields either fall outside the claimed arc range, lack a continuous longitudinal corridor of sufficient length, or are not dimensioned for the percutaneous endoscopic spinal application disclosed herein, and accordingly do not provide this accommodation capability for the claimed application.

The open sector 26 extends along at least 50% of the total length of the elongated body 12, and in preferred embodiments extends continuously from the proximal end 14 to the distal end 16. An open sector extending along at least 50% of the body length provides the clinically meaningful corridor length required for controlled implant delivery under endoscopic visualization; a shorter opening does not provide sufficient working length for this purpose.

In preferred embodiments, the substantially fixed cross-sectional geometry of the elongated body 12 is characterized by transverse cross-sectional dimensions—including D_i, W, and the arc angle—that deviate by no more than about 5% from their nominal unloaded values under operative loads. This 5% threshold is an illustrative quantitative benchmark for one preferred embodiment and is not intended to define the outer boundary of the term “substantially fixed cross-sectional geometry” as used in the independent claims. The functional distinction between the non-expandable retractor of this disclosure and expandable devices lies in the absence of any designed expansion mechanism, not in any particular numerical deviation threshold. Expandable retractors, by contrast, are designed to undergo intentional dimensional changes that far exceed this illustrative benchmark—typically on the order of 50% to 200% or more—through active mechanical radial expansion.

### III. Open Sector—Structural Basis for Implant Accommodation

Referring to FIG. 2A, the open sector 26 forms a region that extends radially beyond the outer circumference of the elongated body 12 without intervening structure. This is a fixed geometric feature: the region exists by virtue of the incomplete circumference of the elongated body 12 and requires no mechanical actuation. The open sector 26 is not a gap created by expansion; it is a pre-existing geometric property of the non-expandable elongated body 12.

Referring to FIG. 3B, when an interbody cage 30 having a maximum transverse dimension D_IMPL satisfying D_i<D_IMPL and D_IMPL less than or equal to D_i+W is introduced into the working channel 20, a portion of the cage 30 exceeding D_i occupies the open sector 26, residing in the region extending radially beyond the outer circumference of the elongated body 12. The cage 30 translates longitudinally through the elongated body 12 with the protruding portion in the open sector 26. This mechanism is independent of any elastic or mechanical property of surrounding tissue; the open sector 26 defines a fixed geometric clearance. The geometric clearance provided by the open sector 26 permits implant passage without reliance upon deformation or displacement of surrounding paraspinal musculature or other soft tissue. The implant accommodation is thus a structural property of the retractor geometry, not a function of tissue compliance, expandability of the retractor walls, or any other variable external to the retractor itself.

### III-A. Tapered and Variable-Diameter Embodiments

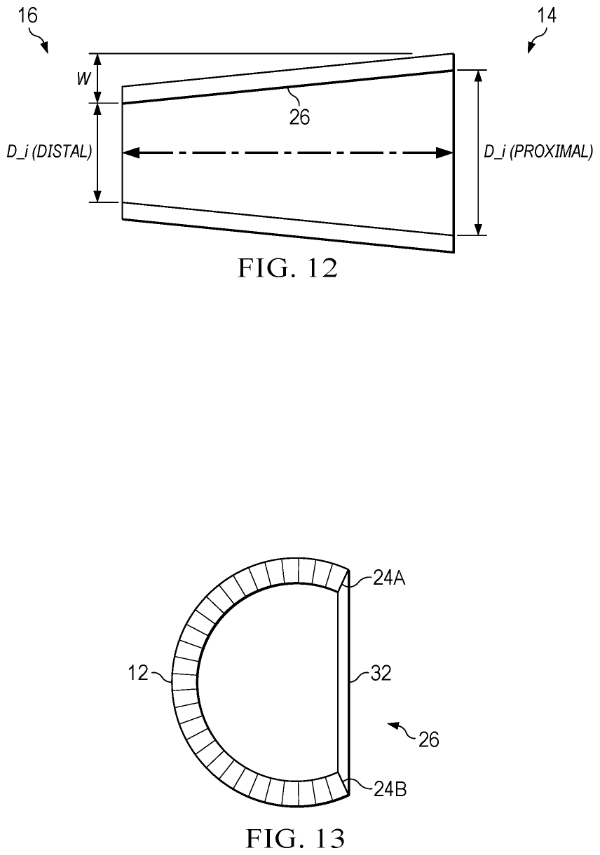

Referring to FIG. 12, in an alternative embodiment, the elongated body 12 has a tapered configuration wherein the internal diameter D_i varies along the longitudinal axis L. In such embodiments, D_i is larger at the proximal end 14 (to facilitate instrument insertion) and tapers to a smaller value at the distal end 16 (to conform to the narrower anatomical corridor at the surgical target). The transverse chord width W correspondingly varies along the longitudinal axis. In tapered embodiments, the arc angle A remains within about 180 degrees to about 300 degrees along a continuous longitudinal portion of the open sector spanning at least 50% of the total length of the elongated body 12, such that the transverse chord width W remains correspondingly within the geometrically determined range of approximately 0.5 times D_i to D_i along that portion. The arc constraint need not be satisfied at every transverse cross-section (e.g., a short distal tip segment may fall outside the range to facilitate docking or anatomical engagement without affecting the implant-accommodation function of the open sector). The open sector 26 in tapered embodiments maintains a continuous longitudinal corridor extending along at least 50% of the body length, such that implant accommodation is preserved throughout the functionally relevant region.

### III-B. Compliant Sheath Embodiments

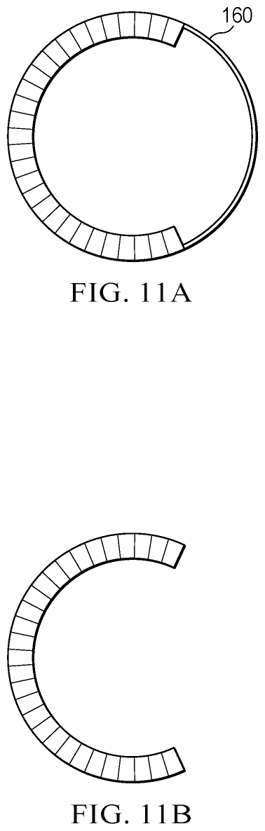

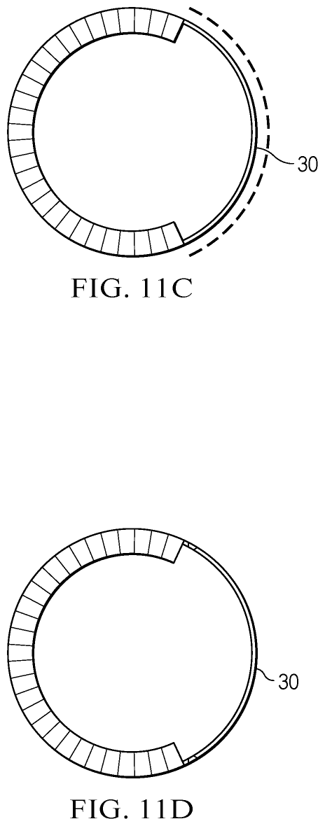

Referring to FIG. 11, in further embodiments, the retractor system includes a compliant sheath 160 positionable over the open sector 26. The compliant sheath 160 may take any of the following forms:

Removable Sheath (FIGS. 11A-11B). In one embodiment, the compliant sheath 160 is a removable elastomeric sleeve or silicone membrane that the user positions over the open sector 26 prior to insertion, providing a temporary barrier against tissue herniation during procedures where the open sector is not actively in use for implant passage. When an implant 30 having D_IMPL greater than D_i is advanced through the elongated body 12, the removable compliant sheath 160 yields laterally upon contact with the protruding portion of the implant (FIG. 11C), permitting passage without impeding longitudinal translation. Alternatively, the surgeon may remove the sheath 160 entirely before implant advancement.

Integral Elastic Membrane. In an alternative embodiment, a thin elastic membrane is bonded or otherwise affixed to the longitudinal edges 24A, 24B across the open sector 26. The elastic membrane stretches and displaces laterally upon contact with the advancing implant, accommodating the protruding portion of the implant without impeding longitudinal translation. The membrane returns to its resting position after the implant has passed.

Frangible Film (FIG. 11D). In a further alternative embodiment, a frangible film—formed from a thin biocompatible polymer, a scored elastomer, or similar material designed to fracture under a predetermined lateral force—spans the open sector 26. When the implant 30 is advanced through the elongated body 12, the frangible film ruptures upon contact with the protruding portion of the implant, eliminating any barrier to passage. The frangible film is designed to fracture cleanly and not generate debris that could migrate to the surgical site.

In all of the foregoing embodiments, the compliant sheath 160—whether removable, integral elastic, or frangible—does not constitute intervening structure as defined herein. None of these barriers impede longitudinal translation of the implant through the open sector. The sheath embodiments address the clinical scenario where the surgeon desires temporary soft tissue containment during the decompression phase but subsequently requires full open-sector access for oversized implant delivery.

### IV. Frangible Bridge Embodiment

Referring to FIG. 13, in one alternative embodiment, the open sector 26 includes one or more bridge members 32 spanning between the first and second longitudinal edges 24A, 24B at discrete axial positions. Each bridge member 32 is frangible (fracturing upon a predetermined lateral force) or removable prior to use. The bridge member 32 does not alter the non-expandable, fixed-geometry nature of the solid wall portion 28.

### V. Atraumatic Edge Profile

Referring to FIG. 2C, the first and second longitudinal edges 24A, 24B each define an atraumatic profile comprising at least one of: a rounded edge with radius r greater than or equal to 0.5 mm; a chamfer between 30 degrees and 60 degrees relative to the inner surface 18; and a flared lip.

### VI. Modular Obturator

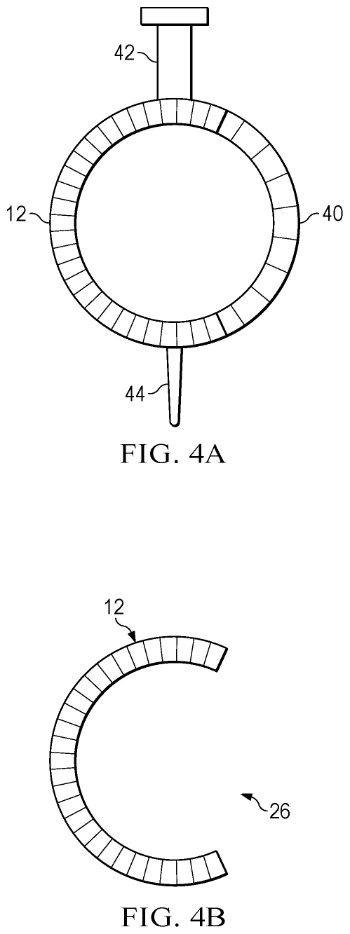

Referring to FIGS. 4A-4B, a modular obturator 40 occupies the open sector 26 during initial percutaneous insertion, creating a flush, continuous 360-degree outer profile that prevents tissue coring during passage through the fascia and musculature. A tapered distal tip 44 facilitates serial tissue dilation. The obturator 40 is withdrawn by its proximal handle 42 after the elongated body 12 is docked at the target anatomy, restoring the open sector 26.

### VII. Robotic Integration—Angular Orientation of the Open Sector

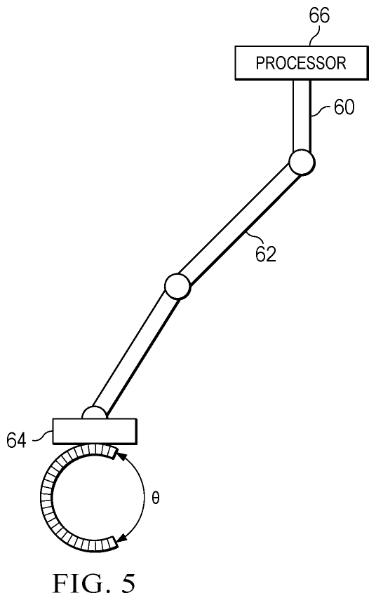

Referring to FIG. 5, a robotic interface feature 64 at the proximal end 14 couples the elongated body 12 to a robotic arm 62 of a surgical robotic system 60. A processor 66 is configured to: (a) receive anatomical imaging data—including preoperative imaging data (such as preoperative MRI or CT) and/or intraoperative imaging data (such as intraoperative CT, O-arm, cone-beam CT, or live fluoroscopy)—and identify the anatomical location of targeted structures including, without limitation, the exiting nerve root, bony landmarks such as the pedicle and lamina, and disc structures; (b) calculate the rotational orientation angle theta of the open sector 26 about the longitudinal axis L that optimizes angular positioning between the open sector 26 and the identified targeted structures; and (c) rotate the elongated body 12 via the robotic arm 62 to orient the open sector 26 at the calculated angular position relative to the targeted structures. The processor's calculation is specifically based on the angular relationship between the open sector 26 and the patient's anatomy —a non-obvious linkage between the fixed open-sector geometry of this device and the robotic system, distinguishing this from general surgical navigation or trajectory planning.

### VIII. Integrated System Features

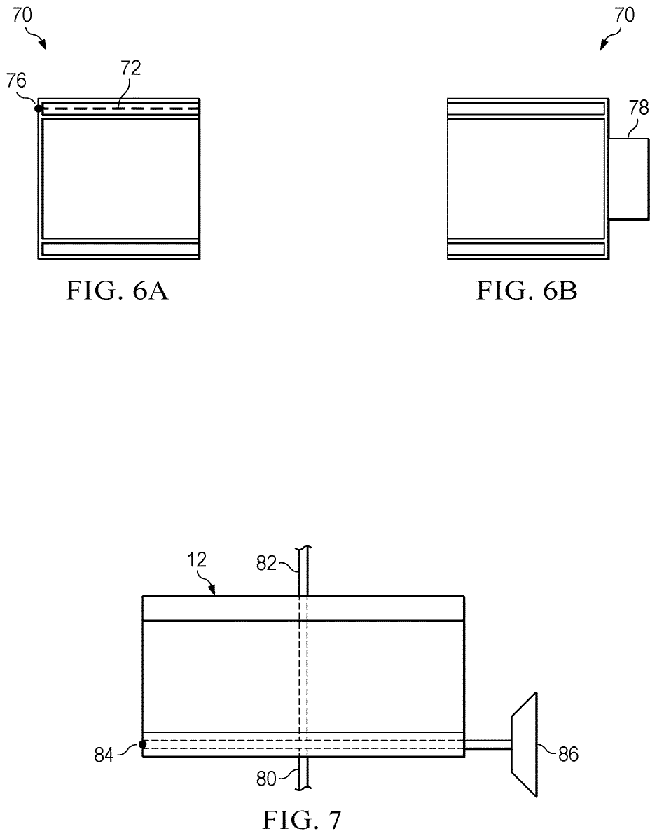

Illumination assembly 70 (FIGS. 6A-6B): At least one optical waveguide 72 integrated longitudinally within the solid wall portion 28 and terminating at a distal light-emitting surface 76, connectable to an external light source; or, alternatively, a modular LED module 78 removably attached to the proximal end 14.

Fluid evacuation mechanism 80 (FIG. 7): A suction lumen 82 extending longitudinally along the elongated body 12, terminating distally at a distal aspiration port 84 and proximally at a vacuum connector 86 configured to interface with external surgical vacuum tubing.

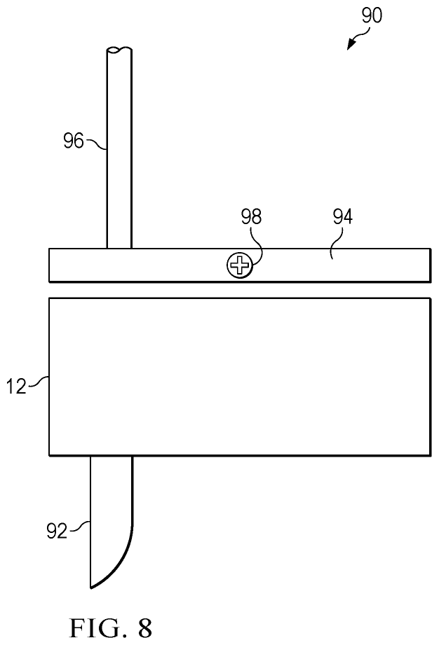

Tool-securing mechanism 90 (FIG. 8): A longitudinal track 94 on the solid wall portion 28 slidably receives the shaft 96 of a secondary surgical instrument. A mechanical locking interface 98—such as a set screw, ratchet, or friction-fit collet—locks the shaft 96 at a selected axial and rotational position, enabling a retractor blade 92 to maintain continuous static retraction of structures while the central working channel 20 and open sector 26 remain accessible for implant delivery.

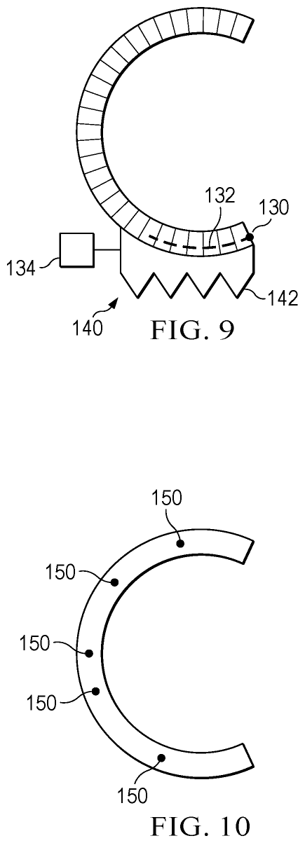

Neuromonitoring electrodes 130 (FIG. 9): At least one stimulation electrode 130 at the distal end 16, positioned adjacent to the open sector 26, interfaces with an intraoperative neuromonitoring (IONM) system via conductive trace 132 and proximal monitoring connector 134. Directional EMG feedback confirms that the open sector 26 is oriented at a safe angular position relative to the traversing and exiting nerve roots before and during implant advancement.

Distal docking surface 140 (FIG. 9): Teeth 142 or micro-serrations on the distal end 16 engage the spinal lamina or facet joint, resisting lateral and longitudinal migration of the elongated body 12 under operative loads.

Radiopaque markers 150 (FIG. 10): An asymmetric arrangement of radiopaque markers 150—such as tantalum pins—embedded in a radiolucent elongated body 12 provides fluoroscopic visualization of the rotational orientation of the open sector 26. The asymmetric marker pattern projects a recognizable, orientation-specific two-dimensional image under fluoroscopy, enabling the surgeon and robotic system to confirm the angular position of the open sector 26 relative to patient anatomy intraoperatively.

### IX. Representative Clinical Operative Sequences

Sequence A—Transforaminal Lumbar Interbody Fusion (TLIF): (1) anatomical imaging data (preoperative MRI/CT and/or intraoperative O-arm or cone-beam CT) is analyzed to determine the optimal open-sector orientation angle theta; (2) a viewing portal incision receives the endoscope; (3) a working portal incision along the pedicle screw trajectory is made; (4) sequential dilators are inserted over a guidewire; (5) the retractor 10, with obturator 40 inserted, is advanced and docked—docking teeth 142 engaging the lamina; (6) the robotic arm 62 rotates the elongated body 12 to angle theta, confirmed fluoroscopically via markers 150 and/or updated via intraoperative imaging; (7) the obturator 40 is withdrawn; (8) the retractor blade 92 is locked in the tool-securing mechanism 90; (9) IONM electrodes 130 confirm safe angular orientation; (10) the oversized interbody cage 30 is advanced—a portion occupying the open sector 26 during longitudinal translation; (11) instruments are removed and pedicle screws are placed through the same corridor.

Sequence B—Endoscopic Spinal Decompression (Without Fusion): (1) anatomical imaging data determines optimal insertion trajectory and open-sector orientation; (2) a viewing portal receives the endoscope; (3) a working portal receives the retractor 10 with obturator 40 inserted; (4) the retractor 10 is docked at the lamina; (5) the obturator 40 is withdrawn; (6) decompression instruments—rongeurs, curettes, high-speed burr—are introduced through the central working channel 20; (7) the open sector 26 is oriented away from neural or other vital structures to provide an additional margin of safety; (8) bone and ligamentous material is removed under endoscopic visualization; (9) the retractor 10 is withdrawn. In this sequence, the open sector 26 serves a protective orientation function and surgical instrument guide rather than an implant-accommodation function, demonstrating the multi-modal clinical utility of the retractor's fixed open-sector geometry.

What is claimed is:

1. A surgical retractor comprising:
an elongated, non-expandable body extending along a longitudinal axis from a proximal end to a distal end, the elongated body having an inner surface defining a central working channel of an internal diameter D_i, and an outer surface;
wherein the elongated body comprises a solid wall portion spanning a continuous arc of about 180 degrees to about 300 degrees about the longitudinal axis, the solid wall portion terminating at a first longitudinal edge and a second longitudinal edge;
wherein the first and second longitudinal edges define an open sector therebetween, the open sector extending along a continuous longitudinal portion spanning at least 50% of a total length of the elongated body, the open sector having a transverse chord width W measured as the straight-line distance between the first and second longitudinal edges in a transverse plane perpendicular to the longitudinal axis; and
wherein the open sector is defined by the absence of any structure between the first and second longitudinal edges that permanently prevents or materially obstructs longitudinal passage of an implant, the open sector forming a region extending radially beyond an outer circumference of the elongated body without intervening structure, and the elongated body maintains a substantially fixed cross-sectional geometry under operative loads without active mechanical expansion.

2. The surgical retractor of claim 1, wherein the substantially fixed cross-sectional geometry is characterized by transverse cross-sectional dimensions of the elongated body, including D_i, W, and an arc angle of the continuous arc, deviating by no more than about 5% from nominal unloaded values under operative loads.

3. The surgical retractor of claim 1, wherein the solid wall portion spans a continuous arc of between 200 degrees and 260 degrees.

4. The surgical retractor of claim 1, wherein the continuous arc spans between about 260 degrees and about 280 degrees, such that the transverse chord width W is between approximately 0.64 multiplied by D_i and approximately 0.77 multiplied by D_i.

5. The surgical retractor of claim 1, wherein the elongated body has the total length of 30 mm to 120 mm, the internal diameter D_i of 5 mm to 35 mm, and an outer diameter of 6 mm to 38 mm, and is formed from a biocompatible material selected from the group consisting of surgical-grade stainless steel, titanium alloy, cobalt-chromium alloy, polyether ether ketone, carbon-fiber-reinforced polyether ether ketone, ultra-high-molecular-weight polyethylene, and combinations thereof.

6. The surgical retractor of claim 1, further comprising a removable obturator configured to occupy the open sector and form a continuous 360-degree outer profile during initial percutaneous insertion, the obturator being removable to restore the open sector after positioning.

7. The surgical retractor of claim 1, further comprising a compliant sheath positionable over at least a portion of the open sector, the compliant sheath being configured to provide a temporary barrier against tissue herniation into the open sector, wherein the compliant sheath is removable, yielding, or frangible such that the compliant sheath displaces, deforms, or ruptures upon contact with a spinal implant during passage through the elongated body without impeding longitudinal translation of the spinal implant through the open sector.

8. The surgical retractor of claim 1, further comprising an illumination assembly comprising at least one of: an optical waveguide extending longitudinally along the solid wall portion and terminating at a distal light-emitting surface; or a modular light source removably attachable to the proximal end of the elongated body.

9. The surgical retractor of claim 1, further comprising a suction lumen extending longitudinally along the elongated body and terminating at a distal aspiration port and a proximal vacuum connector configured to interface with an external vacuum source.

10. The surgical retractor of claim 1, further comprising a robotic interface at the proximal end configured to couple to a robotic arm, the robotic interface being configured to operate with a processor that is configured to receive anatomical imaging data comprising preoperative imaging data, intraoperative imaging data, or both, identify one or more targeted anatomical structures therefrom, calculate a rotational orientation angle of the open sector about the longitudinal axis relative to the identified targeted structures, and rotate the elongated body via the robotic arm to orient the open sector at the calculated angle.

11. The surgical retractor of claim 1, wherein the elongated body is composed primarily of a radiolucent material and comprises an asymmetric arrangement of radiopaque markers embedded therein, the arrangement configured to indicate the rotational orientation of the open sector when viewed under fluoroscopic imaging.

12. The surgical retractor of claim 1, further comprising at least one stimulation electrode at the distal end of the elongated body adjacent to the open sector, the at least one stimulation electrode configured to interface with an electromyography system to provide directional neuromonitoring feedback relative to a rotational orientation of the open sector.

13. The surgical retractor of claim 1, further comprising a textured docking surface at the distal end of the elongated body, the textured docking surface comprising a plurality of teeth or micro-serrations configured to engage a spinal lamina or facet joint to resist migration of the elongated body under operative loads.

14. The surgical retractor of claim 1, wherein the open sector extends substantially the full length of the elongated body from the proximal end to the distal end.

15. The surgical retractor of claim 1, wherein the elongated body has a tapered configuration such that the internal diameter D_i varies along the longitudinal axis from a larger proximal internal diameter to a smaller distal internal diameter, and wherein the continuous arc remains within about 180 degrees to about 300 degrees along the continuous longitudinal portion of the open sector spanning at least 50% of the total length of the elongated body.

16. A spinal surgical system comprising:
a surgical retractor comprising a non-expandable elongated body having an inner surface defining a central working channel of an internal diameter D_i, a solid wall portion spanning a continuous arc of about 180 degrees to about 300 degrees about a longitudinal axis and terminating at first and second longitudinal edges defining an open sector, wherein, along a continuous longitudinal portion of the open sector spanning at least 50% of a total length of the elongated body, the open sector has a transverse chord width W measured between the first and second longitudinal edges, the open sector forming a region extending radially beyond an outer circumference of the elongated body without intervening structure, the elongated body maintaining a substantially fixed cross-sectional geometry under operative loads without active mechanical expansion; and
an interbody spinal implant having a maximum transverse dimension D_IMPL, wherein D_IMPL is greater than D_i and D_IMPL is less than or equal to D_i plus W, such that a portion of the interbody spinal implant is positionable within the open sector during passage through the elongated body, and wherein the interbody spinal implant is configured to be advanced longitudinally through the elongated body while the portion of the interbody spinal implant occupies the open sector, without mechanical actuation of the elongated body.

17. The spinal surgical system of claim 16, further comprising a surgical robotic system having a robotic arm coupled to a proximal end of the elongated body and a processor configured to receive anatomical imaging data comprising preoperative imaging data, intraoperative imaging data, or both, identify one or more targeted anatomical structures therefrom, calculate a rotational orientation angle for the open sector relative to the identified targeted structures, and rotate the elongated body via the robotic arm to orient the open sector at the calculated angle prior to passage of the interbody spinal implant.

18. A method of using a surgical retractor, comprising:
providing a surgical retractor comprising a non-expandable elongated body having a central working channel of an internal diameter D_i, a solid wall portion spanning a continuous arc of about 180 degrees to about 300 degrees, and an open sector defined between first and second longitudinal edges, wherein the open sector extends along a continuous longitudinal portion spanning at least 50% of a total length of the elongated body and has a transverse chord width W measured between the first and second longitudinal edges;
inserting the elongated body into a percutaneous surgical access corridor;
orienting the elongated body to position the open sector at a predetermined angular position relative to one or more targeted anatomical structures, away from neural or other vital structures; and
advancing a spinal implant having a maximum transverse dimension D_IMPL, wherein D_IMPL is greater than D_i and D_IMPL is less than or equal to D_i plus W, through the elongated body such that a portion of the spinal implant occupies the open sector during longitudinal translation, without mechanical expansion of the elongated body.

19. The method of claim 18, further comprising inserting a removable obturator into the open sector prior to inserting the elongated body to create a continuous 360-degree outer profile, and removing the obturator after positioning the elongated body at the surgical target site to restore the open sector.

20. The method of claim 18, further comprising: coupling the elongated body to a robotic arm of a surgical robotic system; determining, by a processor from anatomical imaging data comprising preoperative imaging data, intraoperative imaging data, or both, an optimal rotational orientation angle for the open sector relative to one or more targeted anatomical structures; rotating the elongated body via the robotic arm to the determined angle; confirming the rotational orientation under fluoroscopic imaging via an asymmetric radiopaque marker pattern embedded in the elongated body; and activating at least one neuromonitoring electrode adjacent to the open sector to verify angular orientation relative to neural structures before advancing the spinal implant.
